(19) United States
(12) Patent Application Publication
(10) Pub. No.: US 2014/0277237 A1
(43) Pub. Date: Sep. 18, 2014

(54) DEVICE BASED OPTIMIZATION OF DEVICE THERAPIES

(71) Applicant: Cardiac Pacemakers, Inc., St. Paul, MN (US)

(72) Inventors: Barun Maskara, Blaine, MN (US); Qi An, Blaine, MN (US); Pramodsingh Hirasingh Thakur, Woodbury, MN (US); Julie A. Thompson, Circle Pines, MN (US)

(73) Assignee: Cardiac Pacemakers, Inc., St. Paul, MN (US)

(21) Appl. No.: 14/187,877

(22) Filed: Feb. 24, 2014

Related U.S. Application Data
(60) Provisional application No. 61/779,155, filed on Mar. 13, 2013.

(51) Int. Cl. A61N 1/365

(52) U.S. Cl. CPC A61N 1/36585
USPC 607/18; 607/17

(57) ABSTRACT

A system may include an external medical device (e.g., a patch) including one or more physiological sensors configured to sense one or more physiological parameters of a subject when the subject is ambulatory. The external medical device may be configured to communicate information related to the sensed one or more physiological parameters for determining and/or modifying at least one cardiac therapy parameter of an implantable medical device (e.g., pacemaker, implantable cardioverter defibrillators, or cardiac resynchronization therapy device). In some situations, an indication or notification may be generated corresponding to the determined and/modified cardiac therapy parameter.

### CLAIM OF PRIORITY

This application claims the benefit of U.S. Provisional Patent Application Ser. No. 61/779,155, filed on Mar. 13, 2013, the benefit of priority of which is claimed hereby, and which is incorporated by reference herein in its entirety.

### BACKGROUND

Ambulatory medical devices include implantable medical devices (IMDs) and wearable medical devices. Some examples of IMDs include cardiac function management (CFM) devices such as implantable pacemakers, implantable cardioverter defibrillators (ICDs), cardiac resynchronization therapy devices (CRTs), and devices that include a combination of such capabilities. The devices can be used to treat patients or subjects using electrical or other therapy or to aid a physician or caregiver in patient diagnosis through internal monitoring of a patient's condition. The devices may include one or more electrodes in communication with one or more sense amplifiers to monitor electrical heart activity within a patient, and often include one or more sensors to monitor one or more other internal patient parameters. The devices may be implantable subcutaneously and include electrodes that are able to sense cardiac signals without being in direct contact with the patient's heart. Other examples of IMDs include implantable diagnostic devices, implantable drug delivery systems, or implantable devices with neural stimulation capability.

Some examples of wearable medical devices include wearable cardioverter defibrillators (WCDs) and wearable diagnostic devices (e.g., an ambulatory monitoring vest). WCDs can be monitoring devices that include surface electrodes. The surface electrodes are arranged to provide one or both of monitoring to provide surface electrocardiograms (ECGs) and delivering cardioverter and defibrillator shock therapy.

Parameters to deliver cardiac therapy can be programmable. To optimize the therapy parameters, the patient typically visits a clinic for one or more follow-up visits. A clinician then performs procedures to determine therapy parameter settings while the patient is at rest. However, it is anticipated that optimum settings for the patient may change when the patient is active and away from the clinic.

### Overview

This document relates generally to systems, devices, and methods that provide electrical pacing therapy to the heart of a patient or subject. In particular it relates to systems, devices, and methods that automate programming of device-based therapy parameters.

A system example can include a wearable medical device and a second medical device. The wearable medical device can include one or more physiological sensor circuits (wherein a sensor circuit is configured to provide a physiological sensor signal that includes physiological information of a subject) and a communication circuit configured to communicate information with a separate second medical device. The second medical device includes a communication circuit configured to communicate information with the wearable medical device, and a control circuit in electrical communication with the communication circuit and including a parameter circuit configured to determine a value of at least one programmable therapy parameter of cardiac therapy using physiological information obtained by the wearable medical device when the subject is ambulatory and communicated from the wearable device, and generate an indication of the determined therapy parameter value.

This section is intended to provide an overview of subject matter of the present patent application. It is not intended to provide an exclusive or exhaustive explanation of the disclosure. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, the various examples discussed in the present document.

FIG. 1 is a flow diagram of a method of operating a medical device system.

FIG. 2 is an illustration of portions of an example of a medical device system.

FIG. 3 shows a block diagram of portions of an example of a medical device system.

FIG. 4 is an illustration of portions of another example of a medical device system.

FIG. 5 is an illustration of portions still another example of a medical device system.

### DETAILED DESCRIPTION

An ambulatory medical device may include one or more of the features, structures, methods, or combinations thereof described herein. For example, an ambulatory cardiac monitor or cardiac stimulator may be implemented to include one or more of the advantageous features or processes described below. It is intended that such a monitor, stimulator, or other implantable, partially implantable, or wearable device need not include all of the features described herein, but may be implemented to include selected features that provide for unique structures or functionality. Such a device may be implemented to provide a variety of therapeutic or diagnostic functions.

This document discusses systems, devices and methods for improved determination of cardiac therapy parameters for a patient or subject. As explained previously herein, re-programming to optimize therapy parameters for device-based cardiac therapy typically involves a follow-up visit to a clinic where settings for various device parameters are determined when a patient is at rest. However, device settings determined for a patient at rest at a clinic may not be optimized for a patient who is active. For example, a patient may be prescribed a device that provides cardiac pacing therapy such as cardiac resynchronization therapy (CRT). Even a change in posture of a patient can have a substantial effect on preloading of the ventricles that might influence the desired setting of therapy parameters such as, among other things, atrio-ventricular (AV) delay, inter-ventricular (VV) delay, choice of a pacing vector, and pacing pulse amplitude. Physiological information collected when a patient is ambulatory (e.g., physically active) can be useful to better optimize programmable therapy parameters.

FIG. 1 is a flow diagram of a method 100 of operating a medical device system to optimize therapy parameters. At block 105, a plurality of physiological sensor signals is sensed from a subject while the subject is ambulatory, or otherwise not at rest, using an external medical device of the medical device system. The external medical device can be an adherable device (e.g., a patch), a wearable device (e.g., the device includes a belt, or is incorporated into a garment), or otherwise external device. A physiological sensor signal includes physiological information related to the subject. In one example, the adherable medical device can be a multi-sensor external patch worn by the subject. This allows physiological information about the subject to be collected when the subject is away from a clinical setting.

At block 110, the physiological information obtained by the external medical device can be communicated to second medical device of the medical device system. Some examples of a second medical device can include a programmer, a communicator, an implantable medical device (e.g., pacemaker, transvenous ICD, subcutaneous ICD, CRT, neurostimulator, diagnostic only device, etc.) or any other suitable device for communicating the external medical device. The communication between the external medical device and the second medical device can be wired or wireless (e.g., radio frequency or magnetic communication). In some case, the second medical device may interrogate the external medical device using wireless telemetry and upload the physiological information into the second medical device. If wired communication is provided, the external medical device may include a serial port or other communication port connectable to the second medical device. In some situations, the external device may be a wearable device that may be removed from the subject during the uploading of the physiological information.

At block 115, a setting for at least one programmable therapy parameter of cardiac pacing therapy can be determined by the second medical device using (at least in part) the physiological information communicated from the external medical device. The second medical device may then generate an indication of the determined therapy parameter setting. For example, the second medical device may use the setting to program a third medical device of the medical device system.

FIG. 2 is an illustration of portions of an example of a medical device system 200. The system includes an external medical device 205 and a second medical device 207. The external medical device 205 may be an adherable patch that may be adhered (e.g., by an adhesive) to the skin of the subject. The adherable medical device 205 may be battery powered and may include a housing that contains an electronics unit. A portion of the housing may include an adhesive to temporarily attach the device to the subject (e.g., attach the device next to the skin). In certain examples, the external medical device 205 may be attached to a belt or strap to allow the external medical device 205 to be worn by the subject. In certain examples, the external medical device is incorporated into a garment (e.g., a vest) worn by the subject.

FIG. 3 shows a block diagram of portions of an example of a medical device system 300 that includes an adherable medical device 305 and a second medical device 307. The adherable medical device 305 can include a plurality of physiological sensors or physiological sensor circuits (350A-3501V).

The one or more physiological sensor circuits can be configured to sense one or more physiological parameters of a subject and, in some cases, provides one or more physiological sensor signals corresponding to the sensed physiological parameters. Some examples of a physiological sensor includes a heart sound sensor, a lung sound sensor, respiration sensor, a posture sensor, a transthoracic impedance sensor, a physical activity sensor, a heart rate sensor, and other physiological sensors. The heart sound sensor may be configured to sense one or more of the subject's heart sounds. A “heart sound” can include a first heart sound (“S1”), a second heart sound (“S2”), a third heart sound (“S3”), a fourth heart sound (“S4”), or any components thereof, such as the aortic component of S2 (“A2”), the pulmonary component of S2 (“P2”), or other broadband sounds or vibrations associated with mechanical activity of the heart, such as valve closures or fluid movement (e.g., a heart murmur, etc.). Heart sounds can also include one or more broadband chest sounds, such as may result from one or more of mitral regurgitation, left ventricle dilation, etc. The heart sound sensor may provide an electrical heart sound signal that includes heart sound information and, in some cases, may vary with time. Some examples of a heart sound sensor include an accelerometer, a microphone, or other suitable heart sound sensor.

The lung sound sensor may provide an electrical “lung sound signal” corresponding to lung sound information. The lung sound signal can include any signal indicative of at least a portion of at least one lung sound of the subject. Some examples of a lung sound sensing circuit include an accelerometer and a microphone. If the adherable medical device includes both a lung sound sensing circuit and a heart sound sensing circuit, signal processing (e.g., signal filtering) can be used to discern the heart sounds and lung sounds from each other.

The respiration sensor may provide an electrical “respiration signal” corresponding to respiration information. One or more respiration parameters can be extracted from the respiration signal. Some examples of a respiration parameter include a respiration rate of the subject, an inter-breath interval of the subject, a measure of variability of respiration rate of the subject, a measure of variability of an inter-breath interval of the subject, a tidal volume of the subject, or a measure of variability of tidal volume of the subject. Some examples of the respiration sensing circuit include a motion sensing circuit (e.g., an accelerometer) that senses motion of the thoracic cavity of the subject, and a thoracic impedance sensing circuit that senses impedance signal changes during respiration.

The posture sensor may provide one or more electrical “posture signals” corresponding to posture information. The posture of the subject can be extracted from posture signals, such as whether the subject is in an upright position, a supine position, a prone position, on his or her left or right side, or if the patient is in a tilted position. Some examples of a posture sensing circuit include a multi-axis accelerometer and a tilt switch. Other physiological signals can be monitored in association with determined posture. In this way, a measure extracted from the physiological signal may only be compared to other measurements of the physiological signal obtained when the subject is in the same known posture (e.g., laying on his or her left side).

The transthoracic impedance sensor can include surface electrodes and provide an electrical “transthoracic impedance signal” corresponding to transthoracic impedance information of the subject. In some examples, a first surface electrode can be included in a housing of the adherable medical device 305 and a second surface electrode can be worn at a second location on the subject.

The physical activity sensor may provide an electrical “activity signal” corresponding to physical activity information of the subject. Some examples of a physical activity sensor include an accelerometer, a vibration sensor, or other physical activity sensor.

The heart rate sensor may provide an electrical “cardiac activity signal” that includes heart rate information of the subject. An example of a heart rate sensor includes electrodes in contact with the skin to detect electrical cardiac activity; similar to an electrocardiogram or EKG. The heart rate sensor may include a peak detector circuit and may provide an indication of heart beats used by the adherable medical device 305 to determine heart rate. Some examples of parameters derived from cardiac activity signal are heart rate, heart rate variability, PR interval, QRS width. The heart rate sensor may be incorporated into the adherable medical device 305 or the heart rate sensor may be separate (e.g., a heart rate monitor) and may communicate heart rate information wirelessly to the adherable medical device 305.

The adherable medical device 305 also includes a communication circuit 355 to communicate information with the second medical device 307. The communication circuit 355 may include a wireless transmitter, receiver, or transceiver to communicate wirelessly with the second medical device 307, such as by radio frequency telemetry, mutual inductance telemetry, or other wireless communication protocol. In some examples, the communication circuit 355 may include a transmitter, receiver, or transceiver for communicating over a hard-wired connection with the second medical device.

The second medical device 307 may include a communication circuit 360 to communicate information with the adherable medical device 305 and a control circuit 365 in electrical communication with the communication circuit 360. The communication circuit 360 may include a transmitter, receiver, or transceiver for communication over a wired or wireless link. The control circuit 365 can be a microprocessor, a digital signal processor, application specific integrated circuit (ASIC), microprocessor, or other type of processor, interpreting or executing instructions in software modules or firmware modules. The control circuit 365 can include other circuits or sub-circuits to perform the functions described. These circuits may include software, hardware, firmware or any combination thereof. Multiple functions can be performed in one or more of the circuits as desired.

The control circuit 365 includes a parameter circuit 370 that determines a value or setting of at least one programmable therapy parameter of cardiac pacing therapy using the physiological information communicated from the adherable medical device 305. The parameter circuit 370 generates an indication of the determined therapy parameter value. The generated indication can be used to program device-based therapy.

FIG. 4 is an illustration of portions of another example of a medical device system 400. The system includes a wearable medical device 405, a second medical device 407 and an implantable medical device or IMD 410. The wearable medical device 405 is shown worn by the patient, and the second medical device 407 shown in the example is a communication system. The communication system includes an external communication device 412 and a remote system 414 that communicates with the external communication device 412 via a network 418 (e.g., the internet, a proprietary computer network, or a cellular phone network). The remote system 414 may include a server 416 remotely located from the external communication device 412 and the subject to perform patient management functions. The external communication device 412 may include a programmer to program therapy parameters of a device-based therapy provided by the IMD 410. In certain examples, the external communication device 412 includes a repeater to communicate programming changes or other communication initiated by the remote system 414.

The IMD 410 includes a communication circuit to communicate information with the second medical device 407. The second medical device 407 communicates with both the IMD and the wearable medical device 405. In the example shown, the second medical device 407 may communicate wirelessly with both the wearable medical device 405 and in the IMD 410. In certain examples, the wearable medical device 405 is removed from the subject and physiological information collected by the wearable medical device 405 is uploaded to the second medical device 407 using a wired connection. In some examples, IMD 410 and wearable medical device 405 may additionally communicate information wirelessly with each other.

FIG. 5 is an illustration of portions another example of a medical device system 500 that includes a wearable medical device 505, a second medical device 507 and a third medical device that is an IMD 510. The IMD 510 can include a therapy circuit that delivers cardiac therapy (e.g., cardiac stimulation) to the subject. Examples of the IMD 510 include, without limitation, a pacemaker, a cardioverter/defibrillator, a cardiac resynchronization therapy (CRT) device, or a combination of such devices. Although the example shown includes an IMD with implantable leads contacting the heart, the IMD can include a subcutaneously implantable device with electrodes that do not directly contact the heart. A subcutaneously implantable device can include diagnostic devices and cardioverter/defibrillators. The second medical device 507 communicates wireless signals with the IMD 510, such as by using radio frequency or other telemetry signals.

The IMD 510 can include an electronic unit coupled by one or more leads 508A-C to the heart as shown in the example. Cardiac leads 108A-C include a proximal end that is coupled to IMD 510 and a distal end, coupled by electrical contacts or “electrodes” to one or more portions of the heart. The electrodes may deliver cardiac pacing therapy to the heart such as bradycardia pacing therapy, or bi-ventricular cardiac resynchronization therapy. In some examples, the electrodes can be used to deliver one or both of cardioversion and defibrillation therapy to the heart. Sensed electrical cardiac signals can be sampled to create an electrogram. An electrogram can be analyzed by the IMD and/or can be stored in the IMD and later communicated to an external device where the sampled signals can be displayed for analysis.

The example shows a right atrial lead 508A, a right ventricular lead 508B and a left ventricular lead 508C. Right atrial (RA) lead 508A includes electrodes (electrical contacts, such as a ring electrode and a tip electrode) disposed in an atrium of the heart for sensing signals, or delivering cardiac pacing therapy, or both, to the atrium.

Right ventricular (RV) lead 508B can include one or more electrodes (such as a tip electrode and a ring electrode) for sensing signals, delivering cardiac pacing therapy, or both sensing signals and delivering cardiac pacing therapy. Lead 508B optionally also includes additional electrodes, such as for delivering atrial cardioversion, atrial defibrillation, ventricular cardioversion, ventricular defibrillation, or combinations thereof to the heart. Such electrodes typically have larger surface areas than pacing electrodes in order to handle the larger energies involved in defibrillation. Lead 508B optionally provides resynchronization therapy to the heart. Resynchronization therapy is typically delivered to the ventricles in order to better synchronize the timing of depolarizations between ventricles.

Left ventricular lead 508C can include electrodes placed in a coronary vein lying epicardially on the left ventricle (LV) via the coronary vein, and may include a ring electrode positioned near the coronary sinus (CS).

In some examples, the IMD 510 may be implantable subcutaneously and include electrodes that are positioned more remote from the heart than from what is shown in the example. A subcutaneous device may sense cardiac signals and deliver therapy using electrodes that do not contact the heart directly. A subcutaneous device may simplify the procedure used to implant the device.

Using the physiological information collected by the wearable medical device 505, the parameter circuit of the second medical device 507 adjusts a setting for at least one programmable parameter of therapy provided by the IMD 510. In some examples, second medical device 507 can combine physiological information collected by the wearable medical device 505, with additional physiological information collected by IMD 510 to determine a setting for at least one programmable parameter of therapy provided by the IMD 510. The settings can be adjusted to optimize a device-calculated metric. The current pacing parameter settings may have been previously uploaded to the IMD by the second medical device 507. If the optimized setting is different from the current setting, the second medical device 507 communicates a change in the at least one pacing parameter to the IMD 510.

The second medical device 507 may execute a test to determine the parameter setting. The second medical device 507 communicates a change in the pacing parameter to be optimized and then initiates sensing of the physiological information by the adherable medical device 505 in association with changing of the pacing parameter. The second medical device 507 may then download the physiological information collected by the adherable medical device during a specified (e.g., programmed) period of time when the subject is ambulatory and the pacing parameter is set to the programmed value. The process may be repeated for several values of the pacing parameter to be optimized. Based on the collected physiological information, the optimized setting for the parameter may be determined. The second medical device 507 may then initiate programming and data collecting to determine optimized setting for other pacing parameters. This may continue for other parameters until the second medical device has determined the optimized settings for the pacing parameters of interest.

The IMD 510 may deliver one or both of bradycardia pacing therapy and cardiac resynchronization therapy. Some examples of a pacing parameter that can be programmed by the second medical device 507 include a pacing pulse magnitude, a time delay between an atrial event and a paced ventricular event (AV delay), or a time delay between a ventricular event and a paced ventricular event (VV delay), or a combination of electrodes used to deliver the cardiac pacing therapy. The second medical device 507 determines the optimum setting of at least one of the pacing pulse magnitude, the AV delay, the VV delay, or the combination of electrodes using the physiological information obtained by the adherable medical device 505.

For instance, the second medical device 507 may program the AV delay of the IMD 510 to different settings while initiating sensing of heart sound signals by the adherable medical device 505. The amplitude of the S1 heart sound can be a proxy measurement for ventricular contractility. Based on the collected heart sound data, the parameter circuit of the second medical device 507 may determine the optimized AV delay as the value of AV delay that corresponded to the highest amplitude of the S1 heart sound. The second medical device 507 may program the AV delay with this optimized value or store the optimized value for alter viewing by a clinician or other user. The user may then determine whether to program the AV delay to this determined AV delay value.

The physiological information obtained by the adherable medical device 505 can be associated with ventricular pressure, cardiac stroke volume, ejection fraction, or ventricular pre-load. The parameter circuit of the second medical device 507 determines an optimum setting for the programmable therapy parameter to optimize a device-calculated metric derived from one or more of ventricular pressure, cardiac stroke volume, and ventricular pre-load. The metric may be derived from one or both of physiological signals sensed by the adherable medical device 505 and physiological measurements determined by the adherable medical device 505 using the physiological signals. The metric may be a composite metric determined using any combination of ventricular pressure, cardiac stroke volume, and ventricular pre-load.

In some examples, the adherable medical device stores a device-calculated metric derived from one or more of ventricular pressure, cardiac stroke volume, and ventricular pre-load and optimized in the clinical setting. The adherable medical device may then be programmed to recalculate the metric at subsequent post-clinic times. A significant excursion of the value of metric from the clinic-determined value can trigger the sending of an indication from the adherable medical device 505 to the second medical device 507 to initiate a re-optimization sequence.

In some examples, the cardiac pacing therapy provided by the IMD 510 includes autonomic stimulation therapy. Autonomic stimulation therapy leads and electrodes are designed for placement to provide therapy to specific areas of the nervous system, including the sympathetic nervous system and the parasympathetic nervous system. The sympathetic nervous system is associated with increased blood flow, heart rate, and increased skeletal muscle blood flow. The parasympathetic nervous system is associated with decreased blood pressure, heart rate, and increased digestion. Some examples of sites for delivery of autonomic stimulation include the spinal cord, the vagus nerve, the azygos vein, the vena cava, the carotid sinus, and cardiac fat pads. One example of a lead to provide neural therapy is described in U.S. Pat. No. 8,000,793, filed May 23, 2008, by Libbus et al., entitled “Automatic Baroreflex Modulation Based on Cardiac Activity,” which is incorporated herein by reference in its entirety.

Some examples of a pacing parameter for autonomic stimulation therapy that can be programmed by the second medical device 507 include the amplitude of a stimulation pulse, the stimulation pulse width, the shape of the stimulation signal, the frequency of the stimulation, the duration of the stimulation, or a combination of electrodes used to deliver the autonomic stimulation therapy. The second medical device 507 may change one or more of the parameters when the adherable medical device is sensing physiological information. The second medical device 507 may then determine an optimum setting for at least one of the stimulation pulse amplitude, the stimulation pulse width, the shape of stimulation, the frequency of stimulation, the duration of stimulation, or the combination of electrodes to deliver the autonomic stimulation therapy using the sensed physiological information.

As explained previously herein, the implantable medical device is an example of a third medical device of a medical device system for improved determination of cardiac therapy parameters for a subject. The third medical device may or may not be implantable. The third medical device may include a plurality physiological sensor circuits that provide a physiological sensor signal that includes physiological information of a subject. For example, the third medical device may be an IMD or a wearable medical device, and the third medical device may include a cardiac signal sensing circuit to provide an electrical signal representative of cardiac activity of the subject. The third medical device may include one or more of a heart sound sensing circuit, a lung sound sensing circuit, a respiration sensing circuit, a posture sensing circuit, a transthoracic impedance sensing circuit, a physical activity sensing circuit and a heart rate sensing circuit.

If the third medical device is implantable, the third medical device may include an intracardiac impedance sensing circuit. An intracardiac impedance sensing circuit may include electrodes placed within a chamber of the heart provide an “intracardiac impedance signal” representative of intracardiac impedance versus time. The electrodes may be placed in a right ventricle of the heart and the measured intracardiac impedance waveform can be signal processed to obtain a measure of the time interval beginning with a paced or spontaneous QRS complex (systole marker) and ending with a point where the impedance signal crosses the zero axis in the positive direction following the QRS complex. The resulting time interval is inversely proportional to the contractility of the heart. Systems and methods to measure intracardiac impedance are described in Citak et al., U.S. Pat. No. 4,773,401, entitled “Physiologic Control of Pacemaker Rate Using Pre-Ejection Interval as the Controlling Parameter,” filed Aug. 21, 1987, which is incorporated herein by reference in its entirety.

The physiological sensors available to the medical device system 500 may be distributed between the adherable medical device 505 and the third medical device. The third medical device and the adherable medical device 505 may together include a combination of a heart sound sensing circuit, a lung sound sensing circuit, a respiration sensing circuit, a posture sensing circuit, an transthoracic impedance sensing circuit, a cardiac signal sensing circuit, an intracardiac impedance sensing circuit, a physical activity sensing circuit and a heart rate sensing circuit.

The third medical device can include a therapy circuit that provides cardiac pacing therapy to the subject, and can include a communication circuit to communicate information with the second medical device 507. The third medical device can also include a control circuit in electrical communication with the plurality of physiological sensors, the communication circuit, and the therapy circuit. The electrical communication allows electrical signals to be communicated among the control circuit, the physiological sensors, the communication circuit and the therapy circuit even though there may be intervening electrical circuits between them.

The control circuit of the third medical device varies a value or setting of at least one programmable parameter related to delivery of the therapy while the patient is ambulatory. The third medical device may deduce the subject is ambulatory (such as by one or more of a physical activity sensor, a posture sensor and time of day), or the second medical device 507 may communicate an indication to the third medical device that the subject is ambulatory. The third medical device obtains physiological information using the physiological sensors when the subject is ambulatory, and obtains the physiological information for multiple settings of the programmable therapy parameter. Thus, in this example the task of varying the programmable therapy parameter can be offloaded from the second medical device to the third medical device. The physiological information obtained for the multiple settings can include physiological information associated with at least one of a heart sound, a lung sound, respiration, posture, heart rate, transthoracic impedance, physical activity level and one or more time intervals between device-detected cardiac events.

The adherable medical device 505 may also be collecting physiological information during this time. The third medical device and the adherable medical device 505 communicate the physiological information obtained by the devices to the second medical device 507. The parameter circuit of the second medical device 507 determines a setting for the programmable therapy parameter using the physiological information obtained by the third medical device and using physiological information obtained by the adherable medical device.

To align the physiological information obtained by the different devices in time, one or both of the adherable medical device 505 and the third medical device may include a timestamp with the physiological information. The parameter circuit of the second medical device 507 aligns the physiological information communicated from the adherable medical device and the physiological information communicated from the third medical device. The optimized setting of the programmable therapy parameter is then determined using the aligned information.

Returning to FIG. 3, in some examples the second medical device 307 is an IMD. The IMD includes a plurality of physiological sensor circuits, a therapy circuit, the communication circuit 360, and the control circuit 365. The therapy circuit may be configured to deliver cardiac pacing therapy to the subject. The cardiac pacing therapy may include, among other things, bradycardia pacing, cardiac resynchronization therapy pacing, and autonomic stimulation therapy. The control circuit 365 initiates delivery of cardiac pacing therapy to the subject. In some examples, the therapy circuit is configured to initiate a delivery of a drug to the subject. The drug therapy may be provided from a reservoir included in the IMD or may be provided by a separate device and drug therapy is initiated by an indication (e.g., a signal) from the IMD.

When in a test mode, the control circuit 365 of the IMD varies a setting of at least one pacing parameter when the subject is ambulatory, and initiates sensing of physiological information by the implantable medical device and by the adherable medical device for multiple settings of the at least one pacing parameter. The control circuit 365 receives sensed physiological information from the adherable medical device via the communication circuit 360. The parameter circuit 370 determines the setting for the varied pacing parameter using physiological information sensed by the implantable medical device and using the physiological information received from the adherable medical device.

Using physiological information collected when a patient is ambulatory may lead to more optimized programmable therapy parameters thereby increasing patient well-being, and the device-based optimization can assist the physician in optimizing the device performance.

### Additional Notes

The above detailed description includes references to the accompanying drawings, which form a part of the detailed description. The drawings show, by way of illustration, specific embodiments in which the disclosure can be practiced. These embodiments are also referred to herein as “examples.” In the event of inconsistent usages between this document and any documents incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. These computer-readable media can include, but are not limited to, hard disks, removable magnetic disks, removable optical disks (e.g., compact disks and digital video disks), magnetic cassettes, memory cards or sticks, random access memories (RAM's), read only memories (ROM's), and the like. In some examples, a carrier medium can carry code implementing the methods. The term “carrier medium” can be used to represent carrier waves on which code is transmitted.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A system comprising:
an external medical device including:
one or more physiological sensors configured to sense one or more physiological parameters of a subject when the subject is ambulatory; and
a communication circuit configured to communicate information related to the sensed one or more physiological parameters with a separate second medical device; and
the second medical device including:
a communication circuit configured to receive the information related to the sensed one or more physiological parameters from the external medical device; and
a control circuit in electrical communication with the communication circuit and including a parameter circuit configured to determine a value of at least one programmable cardiac therapy parameter using the information related to the sensed one or more physiological parameters received from the external medical device.

2. The system of claim 1, wherein the external medical device is one of: adherable on the subject, wearable by the subject, or incorporated into a garment.

3. The system of claim 1, wherein the second medical device includes at least one of a medical device communicator or a medical device programmer, and wherein the control circuit of the second medical device is configured to generate an indication of the determined therapy parameter value.

4. The system of claim 1, including an implantable medical device separate from the second medical device, wherein the implantable medical device includes:
a communication circuit configured to communicate with the second medical device; and
a therapy circuit configured to deliver at least one of cardiac pacing therapy or cardiac resynchronization therapy to the subject;
wherein the second medical device is configured to:
communicate a change in at least one pacing parameter to the implantable medical device;
initiate sensing by of the physiological information by the external medical device for multiple values of the at least one pacing parameter; and
determine a setting of the at least one pacing parameter using the physiological information sensed by the external medical device.

5. The system of claim 4, wherein the second medical device is configured to:
communicate, to the implantable medical device, a change to at least one of a pacing pulse magnitude, a time delay between an atrial event and a paced ventricular event (AV delay), or a time delay between a ventricular event and a paced ventricular event (VV delay), or a combination of electrodes used to deliver the cardiac pacing therapy; and
determine an optimum setting of the least one of the pacing pulse magnitude, the AV delay, the VV delay, or the combination of electrodes using the physiological information obtained by the external medical device.

6. The system of claim 1, including an implantable medical device separate from the second medical device, wherein the implantable medical device includes:
a communication circuit to communicate information with the second medical device; and
a therapy circuit configured to deliver autonomic stimulation therapy to the subject,
wherein the second medical device is configured to:
communicate, to the implantable medical device, a change to at least one of a stimulation pulse amplitude, a stimulation pulse width, a shape of stimulation, a frequency of stimulation, a duration of stimulation, or a combination of electrodes used to deliver the autonomic stimulation therapy when the external medical device is sensing physiological information; and
determine an optimum setting of the at least one of the stimulation pulse amplitude, the stimulation pulse width, the shape of stimulation, the frequency of stimulation, the duration of stimulation, or the combination of electrodes to deliver the autonomic stimulation therapy using the sensed physiological information.

7. The system of claim 1,
wherein the external medical device is configured to communicate, to the second medical device, physiological information associated with at least one of ventricular pressure, cardiac stroke volume, ejection fraction, or ventricular pre-load, and
wherein the parameter circuit is configured to adjust the programmable therapy parameter to optimize a device-calculated metric derived from one or more of ventricular pressure, cardiac stroke volume, ventricular pre-load.

8. The system of claim 1,
wherein the physiological sensor circuits include one or more of a heart sound sensing circuit configured to provide a sensed heart sound signal representative of mechanical cardiac activity, a lung sound sensing circuit, a respiration sensing circuit, a posture sensing circuit, an transthoracic impedance sensing circuit, a physical activity sensing circuit and a heart rate sensing circuit, and
wherein the external medical device is configured to communicate, to the second medical device, sensed physiological information associated with at least one of a heart sound of the subject, a lung sound of the subject, respiration of the subject, posture of the subject, transthoracic impedance of the subject, activity level of the subject or heart rate of the subject.

9. The system of claim 1, including a third medical device separate from the second medical device, wherein the third medical device includes:
a plurality of physiological sensor circuits;
a communication circuit to communicate information with the second medical device;
a therapy circuit configured to deliver cardiac therapy to the subject; and
a control circuit in electrical communication with the plurality of physiological sensors, the communication circuit, and the therapy circuit, wherein the control circuit is configured to initiate delivery of cardiac therapy to the subject and, when in a test mode, the control circuit is configured to:
vary a setting of the at least one programmable therapy parameter when the subject is ambulatory;
obtain physiological information when the subject is ambulatory and for multiple settings of the at least one programmable therapy parameter; and
communicate the physiological information to the second medical device,
wherein the parameter circuit of the second medical device is configured to determine a setting for the at least one programmable therapy parameter using the physiological information obtained by the third medical device and using physiological information obtained by the external medical device.

10. The system of claim 9,
wherein the physiological sensor circuits of the third medical device include one or more of a heart sound sensing circuit configured to provide a sensed heart sound signal representative of mechanical cardiac activity, a lung sound sensing circuit, a respiration sensing circuit, a posture sensing circuit, an transthoracic impedance sensing circuit, an intracardiac impedance sensing circuit, a physical activity level sensing circuit and a cardiac signal sensing circuit,
wherein the physiological information obtained by the third medical device when the subject is ambulatory and for multiple settings of the at least one pacing parameter includes physiological information associated with at least one of a heart sound, a lung sound, respiration, posture, heart rate, transthoracic impedance, intracardiac impedance, physical activity level and one or more time intervals between device-detected cardiac events.

11. The system of claim 9, wherein the parameter circuit of the second medical device is configured to align the physiological information communicated from the external medical device and the physiological information communicated from the third medical device.

12. The system of claim 1, wherein the second medical device is an implantable medical device, wherein the implantable medical device includes:
a plurality of physiological sensor circuits;
a therapy circuit configured to deliver cardiac pacing therapy to the subject;
the communication circuit configured to communicate information with the external medical device; and
the control circuit, wherein the control circuit is configured to initiate delivery of cardiac pacing therapy to the subject and, when in a test mode, the control circuit is configured to:
vary a setting of at least one pacing parameter when the subject is ambulatory;
initiate sensing of physiological information by the implantable medical device and by the external medical device for multiple settings of the at least one pacing parameter; and
receive sensed physiological information from the external medical device,
wherein the parameter circuit is configured to determine the setting for the at least one pacing parameter using physiological information sensed by the implantable medical device and using the physiological information received from the external medical device.

13. A method of operating a medical device system, the method comprising:
sensing, by an external medical device of the medical device system, one or more physiological parameters of a subject when the subject is ambulatory;
communicating physiological information related to the sensed one or more physiological parameters obtained by the external medical device to a second medical device of the medical device system; and
determining, by the second medical device, a setting of at least one programmable cardiac therapy parameter using the communicated physiological information related to the sensed one or more physiological parameters.

14. The method of claim 13, including:
providing, by an implantable medical device of the medical device system that is separate from the second medical device, at least one of cardiac pacing therapy or cardiac resynchronization therapy to the subject, wherein the at least one programmable therapy parameter includes at least one pacing parameter; and
initiating a change in the at least one pacing parameter using the second medical device, wherein the physiological information is obtained by the external medical device for multiple values of the at least one pacing parameter.

15. The method of claim 14, including:
varying, by the second medical device, at least one of a pacing pulse magnitude, a time delay between an atrial event and a paced ventricular event (AV delay), a time delay between a ventricular event and a paced ventricular event (VV delay), or a combination of electrodes used to deliver the cardiac pacing therapy when the external medical device is obtaining physiological information, and
wherein determining a setting for the at least one pacing parameter includes adjusting a setting of the least one of the pacing pulse magnitude, the AV delay, the VV delay, or the combination of electrodes using the communicated physiological information obtained by the external medical device.

16. The method of claim 13, including:
providing, by an implantable medical device of the medical device system that is separate from the second medical device, autonomic stimulation therapy;
varying, by the second medical device, at least one of a stimulation pulse amplitude, a stimulation pulse width, a shape of a stimulation signal, a frequency of stimulation, a duration of stimulation, or a combination of electrodes used to provide the autonomic stimulation therapy when the external medical device is obtaining physiological information, and
wherein determining a setting for at least one programmable therapy parameter includes determining an optimum setting of the least one of the stimulation pulse amplitude, the stimulation pulse width, the shape of stimulation, the frequency of stimulation, the duration of stimulation, or the combination of electrodes used to provide the autonomic stimulation therapy using the communicated physiological information obtained by the external medical device.

17. The method of claim 13,
wherein communicating physiological information obtained by the external medical device includes communicating physiological information of the subject associated with at least one of ventricular pressure, cardiac stroke volume, ejection fraction or pre-loading of a ventricle, and
wherein determining a setting for at least one programmable therapy parameter includes determining an optimum setting for the programmable therapy parameter to optimize a device-calculated metric derived from one or more of ventricular pressure, cardiac stroke volume, ejection fraction or pre-loading of the ventricle.

18. The method of claim 13, wherein communicating physiological information obtained by the external medical device includes communicating physiological information associated with at least one of a heart sound of the subject, a lung sound of the subject, respiration of the subject, posture of the subject, transthoracic impedance, activity level of the subject and heart rate of the subject obtained by the external medical device.

19. The method of claim 13,
sensing, by a third medical device of the medical device system that is separate from the second medical device, a plurality of physiological sensor signals when the subject is ambulatory; and
providing, by the third device, cardiac therapy to the subject and varying the at least one programmable parameter of cardiac therapy when the subject is ambulatory, wherein the sensing by the external device and the implantable device occurs for multiple settings of the at least one programmable therapy parameter of cardiac therapy,
wherein communicating physiological information includes communicating physiological information from both the external medical device and the third medical device to the second medical device, and
wherein determining a setting includes determining, by the second medical device, the setting for the at least one programmable therapy parameter of cardiac therapy using the physiological information communicated by the external medical and using the physiological information communicated by the third medical device.

20. The method of claim 19, wherein communicating physiological information obtained by the third medical device includes communicating physiological information of the subject associated with at least one of a heart sound, a lung sound, respiration, posture, heart rate, transthoracic impedance, intracardiac impedance, activity level and one or more time intervals between device-detected cardiac events.
